(19) United States
(12) Patent Application Publication
(10) Pub. No.: US 2026/0060191 A1
(43) Pub. Date: Mar. 5, 2026

(54) DEVICE AND METHOD FOR HOST-INDUCED OVER-EXPRESSING OF ARBUSCULAR MYCORRHIZAL FUNGAL GENES

(71) Applicant: South China Agricultural University, Guangzhou (CN)

(72) Inventors: Wentao HU, Guangzhou (CN); Zhilin ZHANG, Guangzhou (CN); Ming TANG, Guangzhou (CN); Lan PAN, Guangzhou (CN); Hui CHEN, Guangzhou (CN)

(21) Appl. No.: 19/270,540

(22) Filed: Jul. 16, 2025

(30) Foreign Application Priority Data
Aug. 30, 2024 (CN) 202411210534.7

(51) Int. Cl. A01G 31/02; A01G 7/06; C12M 1/00; C12M 1/12; C12N 1/14; C12R 1/645

(52) U.S. Cl. CPC A01G 31/02; A01G 7/06; C12M 23/34; C12M 25/04; C12M 25/14; C12N 1/145; C12R 2001/645

(57) ABSTRACT

Disclosed in the present application is a device and method for host-inducing over-expressing of arbuscular mycorrhizal fungal genes, the device including a mycorrhizal chamber and a hyphal chamber arranged above and below, wherein a bottom of the mycorrhizal chamber is defined with hyphal holes, and a support mesh and a nylon mesh are provided on the hyphal holes, allowing the hyphal to pass through but limiting the passage of a plant root system. The nurse plants were cultured in the mycorrhizal chamber and inoculated with an AM fungal microbial inoculum and watered with a low-phosphorus nutrient solution; a high-phosphorus nutrient solution was injected into the hyphal chamber. Subsequently, a target host plant over-expressing the AM fungal gene is introduced such that the target host plant is joined into the symbiotic network with a high speed of nutrients exchange with the AM fungal hyphae.

### CROSS-REFERENCE TO RELATED APPLICATIONS

The present application is based on and claims the priority benefits of China application No. 202411210534.7, filed on Aug. 30, 2024. The entirety of China application No. 202411210534.7 is hereby incorporated by reference herein and made a part of this specification.

### TECHNICAL FIELD

The present application relates to the field of genetic modification of fungal genes, and in particular to a device and method for host-inducing over-expressing of arbuscular mycorrhizal fungal genes.

### BACKGROUND ART

Arbuscular mycorrhizal (AM) fungi can establish beneficial symbionts with most vascular plants on land, help the host plants absorb water and mineral nutrients, and improve the stress tolerance of the host plants. Because AM fungi cannot survive alone, they must establish a symbiotic relationship with the host to complete their own life cycle. Therefore, the abundant AM fungal propagules are produced by employing the symbiotic association between AM fungi and various mycorrhizal plants, which can then be used to infect plants, resulting in better yield and quality improvement of the plants.

In recent years, with the in-depth study on the symbiotic mechanism of AM fungi and plants, researchers found that AM fungi initiated a series of fungal signaling pathways and some fungal genes involved in the process of improving the stress-tolerance of the host plants, that is, these transcription factors and functional genes of AM fungi played an indispensable role in the synergistic stress-tolerance process of AM fungi symbionts. In order to further explore the specific role of these genes, researchers began to apply the research methodologies of plant gene function to analyze the heterologous functional characterization of these transcription factors or functional genes. At the same time, with the in-depth study of symbiotic interaction and pest infestation of host plants, scientific researchers have found that small RNAs can be exchanged at the symbiotic interface or the interface where pathogens interact with host plants. Subsequently, the technology of host-induced gene silencing (HIGS) came into being. HIGS has been proven to silence the genes in pathogens, pests, and AM fungi when the pathogens parasitize the host plant, the pests (including insects and nematodes) infect the host, and the AM fungi symbiosis with the host plant, i.e., using small RNA shuttled through the interactive interface to achieve RNA interference, thereby specifically reducing or silencing the gene expression in pathogens, pests or AM fungi, and achieving gene silencing in these organisms. In the process of AM symbiotic interaction, only a small amount of small RNAs shuttle through the interaction interface, while a small amount of small RNAs with a hairpin structure based on the principle of RNA interference silencing can effectively achieve the target gene silencing, so as to ensure the success of HIGS technology in the fungal target gene silencing in the symbiotic process of AM fungal and host plants, and then study the impact of target gene silencing on the AM fungi symbiont.

Since AM fungi alone cannot survive stably, AM fungal strains over-expressing AM fungal genes cannot be obtained by conventional technical methods. At present, the silencing of target genes of AM fungi can be achieved by HIGS technology, but there is no application of over-expressing of AM fungal target genes. This may be due to the fact that only a small amount of RNA shuttle through the symbiotic interface between AM fungi and plants, which makes it difficult for conventional HIGS strategy to achieve over-expressing of AM fungal genes in the fungi, i.e., there is no efficient technology to achieve efficient shuttling of RNA molecules through the symbiotic interface and accumulation of a large amount of RNA molecules in the fungi. Patent CN106190944A discloses a method for enrichment of arbuscular mycorrhizal fungal spores by layered culture, which involves dividing a seedling culture container into a lower layer accessible to hyphae, a middle layer for blocking water and fertilizer and root system and an upper layer for growing the root system of a plant, enhancing colonization of AM fungi in the root system and hyphae proliferation and sporulation in the lower layer by blocking the uptake of nutrients by the root system and thereby enhancing the dependence of the plant on the nutrient transfer of AM fungi, but the lower layer is not provided with continuous water and fertilizer supply, does not have a water vapor pressure condition which is favorable for the growth of the hypha and does not establish a mechanism for efficiently and quickly exchanging the nutrients between host plants carrying AM fungi genes and AM fungi hypha networks, which limits hyphal growth and makes it difficult for host plants carrying AM fungal genes to quickly join the AM fungal symbiotic network for high-speed nutrient exchange with plant, i.e., it is difficult to achieve host-induced over-expressing of AM fungal gene in AM fungi. Patent CN110476713A discloses a device and method for continuously collecting AM fungal hyphae. The device uses a mycorrhizal chamber and a hyphal chamber respectively placed on the left and right, inverts planting plants to use the gravitropism of root system to avoid directly absorbing high phosphorus nutrients, so as to improve the symbiotic relationship between AM fungi and plants and facilitate the collection of AM fungal hyphae. However, the inverted growth mode is not conducive to the photosynthesis of plants, thus limiting the growth of the host plants, and thus not conducive to the exchange of the photosynthates of the host plants and the nutrients supplied by AM fungi. It is difficult to achieve host-inducing over-expressing of AM fungal genes in AM fungi because of the inability to achieve high-speed nutrient exchange in the symbiotic interface of arbuscule.

### SUMMARY

It is a first object of the present application to overcome the above drawbacks and disadvantages of the prior art and to provide a device for host-inducing over-expressing of AM fungal genes.

It is a second object of the present application to provide a method for accelerating nutrient exchange between AM fungi and a target host plant at a symbiotic interface and for achieving host-inducing over-expressing of AM fungal genes in the AM fungi based on an HIGS strategy using the device described above.

The above objects of the present application are achieved by the following technical solutions.

The present application provides a device for host-inducing over-expressing of AM fungal genes, the device includes an upper layer and a lower layer, the upper layer is a mycorrhizal chamber and the lower layer is a hyphal chamber; a bottom of the mycorrhizal chamber is defined with a plurality of hyphal holes, through which hyphae can reach the hyphal chamber to absorb nutrients, a support mesh is provided on the hyphal holes, and a nylon mesh is provided on the support mesh, and a pore size of the nylon mesh allows the hyphae to pass through but limits the passage of the plant root system.

The device of the present application can accelerate the nutrient exchange between AM fungi and the target host plant at the symbiotic interface, establish a stable symbiotic relationship, achieve host-inducing over-expressing of AM fungal genes in AM fungi based on the HIGS strategy, and lay a solid foundation for utilizing key genes of AM fungi to enhance the stress-tolerance in their symbiotic systems in the future.

The device of the present application includes a mycorrhizal chamber and a hyphal chamber arranged above and below, wherein the mycorrhizal chamber is used for the co-cultivation of the target host plant and AM fungi, and the hyphal chamber is used for providing continuous water and fertilizer environment convenient for the AM fungal hypha to absorb; the bottom of the mycorrhizal chamber is defined with hyphal holes, and a support mesh and a nylon mesh are provided on the hyphal holes, the above-mentioned design allows the hyphal to pass through but limits the passage of the plant root system. The above-mentioned superimposed mycorrhizal chamber and hyphal chamber can provide stable water vapor pressure conditions, thus facilitating the AM fungal hyphae spreading from the mycorrhizal chamber to the hyphal chamber, continuously absorbing water and fertilizer from the hyphal chamber and performing high-speed nutrient exchange with the plant root system in the mycorrhizal chamber, i.e., forming a stable symbiotic relationship, at the same time accelerating the nutrient exchange between the AM fungi and the target host plant at the symbiotic interface, facilitating a large number of RNA molecules in the root system shuttle through the symbiotic interface. Firstly, the device is utilized to cultivate the nurse plants in the mycorrhizal chamber, inoculate the AM fungal inoculum and irrigate the low-phosphorus nutrient solution in the mycorrhizal chamber, and the high-phosphorus nutrient solution is injected in the mycelium chamber, so that the reciprocal symbiosis between the AM fungi and the nurse plants is enhanced by utilizing the characteristic that only the hyphae absorbs the high-phosphorus nutrient solution in the hyphal chamber, so as to establish the symbiotic network of high-speed nutrient exchange between AM fungi and nurse plants. Then, the hairy root-transformed host plants overexpressing AM fungal genes were introduced. By reducing the competitive growth potential of the original nurse plants, the target host plants were integrated into the symbiotic network established by AM fungi and nurse plants with high-speed nutrient exchange and formed a stable symbiotic relationship with the AM fungal hyphal network. Under this symbiotic relationship, the over-expressing mRNA in the root system of the target host plant entered into the interior of AM fungi through the arbuscule interface and accumulated in excess, thereby achieving over-expressing. This method provides a new way for the over-expressing of AM fungal genes.

Further, the pore size of the nylon mesh is 40 to 45μm.

Further, the support mesh may have a pore size of 0.5 to 0.8 cm.

Further, the support mesh is made of a hard material having a space gap of 0.5 to 0.8 cm.

Further, a pore size of the hyphal holes is 2 to 5 cm.

Further, the hyphal chamber is provided with a support column for supporting the mycorrhizal chamber.

Preferably, the mycorrhizal chamber and the hyphal chamber are formed by overlapping two identical plastic hard white rectangular containers, and a plurality of support columns made of the hard material are provided in the hyphal chamber.

Preferably, the hard material is plastic.

The present application further provides a method for accelerating nutrient exchange between AM fungi and host plant at a symbiotic interface and for achieving host-inducing over-expressing of AM fungal genes in the AM fungi based on the HIGS strategy using the device described above, including the following steps:

S1, adding the sterilized substrate into the mycorrhizal chamber of the above-mentioned devices, and adding the nutrient solution for hyphae growth into the hyphal chamber;

S2, planting nurse plants in the mycorrhizal chamber and inoculating an AM fungal inoculum, watering the nutrient solution for plant growth into the mycorrhizal chamber, and performing symbiotic co-culture to establish a stable symbiotic relationship between the AM fungi and the nurse plants, and thereby achieving a purpose of accelerating nutrient exchange in the symbiotic relationship between the AM fungi and the nurse plants;

S3, using a hairy root-transformed host plants over-expressing AM fungal genes as the target host plant, transplanting the target host plant to the mycorrhizal chamber, and subtracting ½ to ¾ of aerial parts of the nurse plants with scissors, performing symbiotic co-culture to establish a symbiotic relationship between the target host plant and the AM fungi, and enabling target host plant root-synthesized mRNA overexpression products to efficiently pass through arbuscule interface during high-speed nutrient exchange at the arbuscule interface between root cortical cells of target host plant and AM fungi, thereby over-expressing in the AM fungi.

Further, a time of the symbiotic co-culture is 2 to 4 months.

Preferably, the time of the symbiotic co-culture is 3 months.

Further, in the steps S2 and S3, conditions of the symbiotic coculture are: culturing under a light intensity of 24000 to 26000 lux, and daily watering of plant seedlings with tap water besides watering of the nutrient solution for plant growth.

Further, the nutrient solution for plant growth in the step S2 is a low-phosphorus nutrient solution; and a phosphate concentration of the low-phosphorus nutrient solution is 80 to 120μM and a pH value of the low-phosphorus nutrient solution is 5.5-5.7.

Preferably, the phosphate concentration of the low-phosphorus nutrient solution is 100 μM and the pH value of the low-phosphorus nutrient solution is 5.6.

Further, the nutrient solution for plant growth in the step S2 is watered every 10 to 20 days.

Preferably, the nutrient solution for plant growth in the step S2 is watered every 15 days.

Further, the nutrient solution for hyphae growth in the step S1 is a high-phosphorus nutrient solution; and a phosphate concentration of the high-phosphorus nutrient solution is 1 to 3 mM and a pH value of the high-phosphorus nutrient solution is 5.1-5.3.

Preferably, the phosphate concentration of the high-phosphorus nutrient solution is 2 mM and the pH value of the high-phosphorus nutrient solution is 5.2.

Further, a liquid level of the nutrient solution for hyphae growth in the step S1 is 1 to 2 cm lower than the bottom of the mycorrhizal chamber.

Further, the concentration of the nutrient solution for hyphae growth in the hyphal chamber is ensured during the symbiotic co-culture and is changed in a timely manner.

Compared to the prior art, the present application has the following beneficial effects:

The superimposed mycorrhizal chamber and hyphal chamber provided in the device of the present application can provide stable water vapor pressure conditions, thus facilitating the AM fungal hyphae spreading from the mycorrhizal chamber to the hyphal chamber, continuously absorbing water and fertilizer from the hyphal chamber and performing a high-speed nutrient exchange with the plant root system in the mycorrhizal chamber, i.e., forming a stable symbiotic effect, synchronously accelerating the nutrient exchange between the AM fungi and the target host plant at the symbiotic interface, facilitating a large number of RNA molecules in the root system shuttle through the symbiotic interface. Further using the device of the present application, firstly, a symbiotic network system is established in which the AM fungi and conventional mycorrhizal plants exchange nutrients at the symbiotic interface at a high speed; then the target host plants were introduced, and through the strategy of reducing the competitive growth potential of the original mycorrhizal plant, the target host plants were rapidly integrated into the symbiotic network of high-speed exchange nutrients with the AM fungi at the symbiotic interface so that the mRNA molecules of the AM fungal gene being over-expressing in the target host plant root could be shuttled into the AM fungi in large amounts, that is to say, the target host plant-induced AM fungal gene over-expressing in AM fungi was realized. The present application overcomes the defects that AM fungal genes are difficult to over-express in the fungi due to the small amount of RNA at the AM symbiotic interface shuttling through the interaction interface using the conventional HIGS strategy, and also lays a solid foundation for utilizing key genes of AM fungi to enhance the tolerance of AM fungal symbionts to environmental stresses in the future. In addition, the device of the present application is simple in construction and convenient in use, and the device can be manufactured manually and at a relatively low cost.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a front cross-sectional view of a device for host-inducing over-expressing of arbuscular mycorrhizal fungal genes.

FIG. 2 is a schematic bottom view of a mycorrhizal chamber of a device for host-inducing over-expressing of arbuscular mycorrhizal fungal genes.

FIG. 3 is a graph showing green fluorescence of the arbuscule structure in the target host plant root. FIG. 3a is a graph showing a signal of Green Fluorescent Protein (GFP) in a living mycorrhizal symbiotic root system (yellow arrow indicates GFP light-emitting position); FIG. 3b is a graph showing a position of the arbuscule structure confirmed by a fluorescent dye (white arrows indicate the position of the arbuscule structure).

FIG. 4 is a graph showing green fluorescence of the arbuscule structure in the target host plant root when the target host plants are planted in a conventional flowerpot to induce the over-expressing of the arbuscular mycorrhizal fungal genes. FIG. 4a is a graph showing a signal of Green Fluorescent Protein (GFP) in the living mycorrhizal symbiotic root system (yellow arrow indicates GFP light-emitting position); FIG. 4b is a graph showing a position of the arbuscule structure confirmed by a fluorescent dye (white arrows indicate the position of the arbuscule structure).

### DETAILED DESCRIPTION

The present application will now be further described with reference to the drawings and specific examples, which are not to be construed as limiting the present application in any way. Unless otherwise indicated, the reagents, methods, and equipment used in the present application are those conventional in the art.

Unless otherwise noted, the reagents and materials used in the following examples are commercially available.

In the description the present application, it should be noted that the orientation or positional relationships indicated by the terms “center”, “upper”, “lower”, “left”, “right”, “vertical”, “horizontal”, “inner”, “outer”, and the like, are based on the orientation or positional relationships shown in the drawings and are merely for convenience in describing the present application and to simplify the description, rather than indicating or implying that the device or element is referred to must have a particular orientation, be constructed and operated in a particular orientation and, therefore, should not be construed as limiting the present application. Furthermore, the terms “first”, “second”, “third”, “fourth”, and the like, are used for descriptive purposes only and are not to be construed as indicating or implying relative importance.

Example 1: A device for host-induced over-expressing of arbuscular mycorrhizal fungal genes

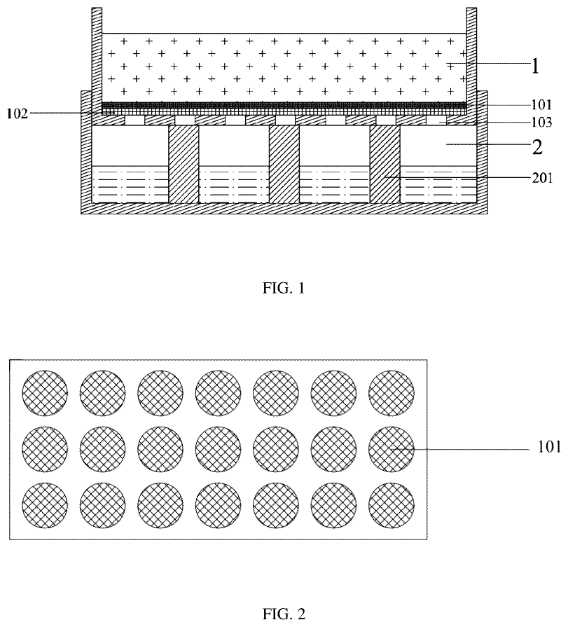

As shown in FIGS. 1 and 2, a device for host-induced over-expressing of arbuscular mycorrhizal fungal genes, wherein the device is an upper layer space and a lower layer space which are formed by overlapping two square containers, and the upper layer space is a mycorrhizal chamber 1 and the lower layer space is a hyphal chamber 2; a bottom of the mycorrhizal chamber 1 is defined with a plurality of equidistant small holes to form hyphal holes 103, hyphae can reach the hyphal chamber 2 through the hyphal holes 103 to absorb nutrients, a support mesh 102 is provided on the hyphal holes 103, and a nylon mesh 101 is provided on the support mesh 102; a support column 201 for supporting the mycorrhizal chamber 1 is provided in the hyphal chamber2. A pore size of the hyphal holes 103 is 2 cm, a mesh gap of the support mesh 102 is 0.5 cm, and a pore size of the nylon mesh is 45 μm.

The device includes a mycorrhizal chamber 1 and a hyphal chamber 2 arranged above and below, wherein the mycorrhizal chamber 1 is used for the co-cultivation of target host plants and the arbuscular mycorrhizal fungi, and the hyphal chamber 2 is used for providing a high-phosphorus water and fertilizer condition which can be absorbed by the hypha; the bottom of the mycorrhizal chamber 1 is defined with hyphal holes 103, a support mesh 102 is provided on the hyphal holes 103, and a nylon mesh 101 is provided on the support mesh 102; since the material of the nylon mesh 101 is relatively soft, when a culture substrate is added on the nylon mesh 101, the nylon mesh 101 is liable to collapse, and therefore a support mesh 102 needs to be arranged below the nylon mesh 101 for supporting; the pore size design of the device allows the passage of hyphae but limits the passage of the plant root system. The above-mentioned superimposed mycorrhizal chamber 1 and hyphal chamber 2 can provide stable water vapor pressure conditions, thus facilitating the AM fungal hyphae spreading from the mycorrhizal chamber 1 to the hyphal chamber 2, continuously absorbing water and fertilizer from the hyphal chamber 2 and performing a high-speed nutrient exchange with the plant root system in the mycorrhizal chamber 1, i.e., forming a stable symbiotic relationship, synchronously accelerating the nutrient exchange between the arbuscular mycorrhizal fungi and the target host plant at the symbiotic interface, facilitating a large number of RNA molecules in the root system shuttle through the symbiotic interface.

Example 2: A method for accelerating the nutrient exchange between arbuscular mycorrhizal fungi and a target host plant at the symbiotic interface and for achieving host-induced over-expressing of arbuscular mycorrhizal fungal genes in the arbuscular mycorrhizal fungi based on the HIGS strategy

The above-mentioned device of the present application is implemented by the following specific method.

S1, Two white rectangular hard plastic containers with length of 38 cm, width of 30 cm, and height of 12 cm were taken, which were cleaned and disinfected with alcohol. 21 small holes with a radius of 2 cm were formed in the bottom of a first container, wherein a distance between the small holes was 1.25 cm, and three rows of 7 small holes were formed in each row and then the first container was used as the mycorrhizal chamber. In the first container formed with small holes, a plastic support mesh with a mesh gap of 0.5 cm was first placed on the bottom of the first container, and then a nylon mesh with a pore size of 45μm was used to cover the bottom of the first container so that the plant root system could not enter the hyphal chamber. Substrates (sand and vermiculite in a volume ratio of 1:1) treated by autoclaving (121° C., 120 min) were filled thoroughly into the mycorrhizal chamber.

S2. The mycorrhizal chamber was stacked with a second hard plastic container, with the mycorrhizal chamber on the top and the second hard plastic container at the bottom. The space formed by overlapping the two containers was the hyphal chamber, and a black hard plastic cylindrical support column (a radius of the bottom surface was 2 cm, and the height was 5 cm) was provided inside the hyphal chamber.

S3. Surface-sterilized seeds of Chrysopogon zizanioides were planted in the mycorrhizal chamber and inoculated with arbuscular mycorrhizal fungi (i.e., Rhizophagus irregularis inoculum), and then co-cultured for 2 months under the conditions of 25000 lux of light intensity and 28° C. of air temperature to ensure good growth of the C. zizanioides, thus enabling AM fungi to establish a stable symbiotic relationship with the C. zizanioides. During the incubation period of two months, the low-phosphorus nutrient solution was poured into the mycorrhizal chamber every 15 days, while the high-phosphorus nutrient solution was injected into the hyphal chamber 2 with a syringe. The liquid level of the nutrient solution in hyphal chamber 2 was 1 to 2 cm lower than the bottom of the mycorrhizal chamber in the step S2. The phosphate concentration in the low-phosphorus nutrient solution, i.e., Hoagland's nutrient solution, was 100μM at pH 5.6, and the phosphate concentration in the high-phosphorus nutrient solution, i.e., Hoagland's nutrient solution, was 2 mM at pH 5.2. After two months, the symbiotic relationship between the arbuscular mycorrhizal fungi (R. irregularis) and the C. zizanioides could be strengthened. That was to say, the root system of C. zizanioides couldn't absorb enough phosphorus nutrients in the mycorrhizal chamber, combining with the barrier of the nylon mesh with the pore size of 45 μm, so the root system of C. zizanioides couldn't absorb the nutrient from the hyphal chamber. Therefore, C. zizanioides necessitates arbuscular mycorrhizal fungal (R. irregularis) hyphae to absorbed a large amount of phosphorus nutrient from the hyphal chamber, thereby R. irregularis supplying phosphorus for nutrient exchange with C. zizanioides and achieving the purpose of accelerating nutrient exchange under the symbiotic relationship between the arbuscular mycorrhizal fungi and plants.

S4. With reference to the prior art document “Boisson-Dernier et al. (2001) Agrobacterium rhizogenes-transformed roots of Medicago truncatula for the study of nitrogen-fixing and endomycorrhizal symbiotic associations”, the RiEFα gene CDS of R. irregularis was cloned into a dicotyledon over-expression vector (i.e., pC1300−35S+eGFP vector) to obtain a constructed vector, and the constructed vector was transferred into an A. rhizogenes ARqua1 strain (hereafter called the transferred A. rhizogenes) for hairy root transformation of Medicago truncatula (M. truncatula):

(1) The seeds of M. truncatula were disinfected with a 5% sodium hypochlorite solution for 20 min, and after disinfection, the seeds were plated in a petri dish containing a germination medium, and then placed in a refrigerator at 4° C. for cultivation for 24 h, and after the cultivation for 24 h, the seeds were placed in an incubator at 25° C. for shading cultivation to inhibit the growth rate of the seeds;

(2) The transferred A. rhizogenes was cultured in a liquid medium with shaking at 30° C. and 200 rpm for 12 h and then taken out and plated on a petri dish containing an LB-containing medium supplemented with antibiotic screen in a clean bench. After plating, the petri dish was inverted and cultured in an incubator at 30° C. for cultivation for 48 h. In the clean bench, the root of M. truncatula (3 mm close to the embryo) was smoothly cut with a blade to produce a wound, and then the wound surface was stained with the transferred A. rhizogenes on the petri dish for infection;

(3) After completion of the infection, the M. truncatula was further plated on a petri dish containing a medium for promoting plant callus budding (budding medium), and placed in an incubator at 18° C. for cultivation for one week.

(4) After one week, the M. truncatula was transferred to an antibiotic screening rooting medium (roots of the M. truncatula should be inserted into the medium) in a clean bench and then cultured in an incubator at 25° C. with a light intensity of 25000 lux to obtain a positive hairy root transgenic plant.

The target host plant M. truncatula (hairy root transformed plant over-expressing the fusion gene of AM fungal RiEFα gene and green fluorescent protein gene) was transplanted into the mycorrhizal chamber 1, and ½ to ¾ of the aerial parts of C. zizanioides were subtracted with scissors to reduce the competitive growth potential of C. zizanioides, thus the competition between the nurse plants C. zizanioides and the target host plant M. truncatula was reduced, and then the establishment of AM symbiotic relationship between the target host plant M. truncatula and the existing AM fungi hyphae network was sped up, and the co-cultivation was performed for one month. In this way, host-inducing over-expressing of the fusion gene of the AM fungal RiEFα gene and green fluorescent protein gene in the fungi could be achieved. That was to say, the target host plant carrying an over-expressed AM fungal gene was joined into a network for symbiotic interaction of an AM fungal hyphal network and a nurse plant for high-speed nutrients exchange established in the above steps, so that the mRNA molecules of the over-expressing fusion gene of the AM fungal RiEFα gene and green fluorescent protein gene in the root system of the target host plant can efficiently pass through the arbuscule interface and be accumulated and over-expressed in the AM fungi in an excessive amount during the process of nutrients exchange at a high-speed at the arbuscule interface between cells in the target host plant root and the AM fungi.

Example 3: Verification of the target host plant-induced over-expressing of AM fungal genes in the fungi.

The target host plant and its roots were dug out together with the root soil in the step S4 of Example 2, the roots of the target host plant were soaked with an ice-water mixture to make the root soil soft, and then the roots were washed by the ice-water mixture for 3 to 4 times. The target host plants were clamped with clean tweezers and collected into a 15 cm diameter glass culture dish. Due to the design of the fusion gene of RiEFα gene and green fluorescent protein gene, whether the green fluorescent protein emits light in the symbiotic arbuscule structure or not can be confirmed by a fluorescent microscope, i.e., the success of over-expressing of the fusion gene of RiEFα gene and green fluorescent protein gene in the arbuscule structure could be verified.

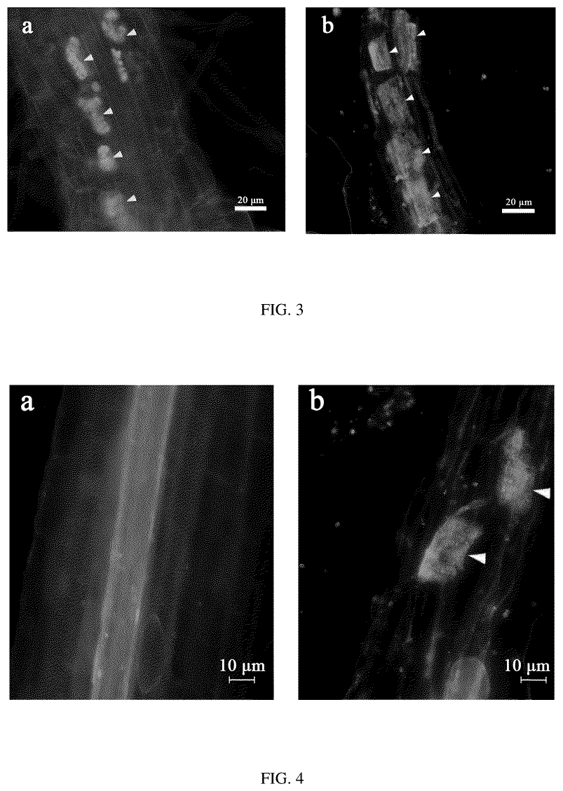

The results were shown in FIG. 3 and the results was observed by the fluorescence microscope. FIG. 3a was a graph showing a signal of Green Fluorescent Protein (GFP) in the living mycorrhizal symbiotic root system, and FIG. 3b was a graph showing a position of the arbuscule structure confirmed by AF488WGA fluorescent dye (NANJING WARBIO, Wheat Germ Agglutinin Alexa Fluor 488, Cat. No. DU-035). The high expression of green fluorescent protein in the symbiotic arbuscule structure could be demonstrated by confirming the green fluorescent signal in the symbiotic arbuscule structure of the living mycorrhizal symbiotic root system according to the corresponding position of the arbuscule structure in FIG. 3a and FIG. 3b, which indicated that the above-mentioned device and method of the present application can achieve the host-induced over-expressing of the fusion gene of RiEFα gene and green fluorescent protein gene in the fungi.

Comparative Example 1: Use of conventional flowerpot planting the target host plant to induce the over-expressing of arbuscular mycorrhizal fungal genes

Surface sterilized seeds of C. zizanioides were planted in conventional flowerpots (square, 20 cm in length and 20 cm in width, i.e., a hyphal chamber not supplied with a high-phosphorus nutrient solution) and inoculated with the arbuscular mycorrhizal fungi (i.e., Rhizophagus irregularis inoculum), and then co-cultured under the culture conditions same as in Example 2.

Positive hairy root transformed plants over-expressing the AM fungal gene (M. truncatula with overexpressing of the fusion gene of AM fungal RiEFα gene and green fluorescent protein gene) were obtained using the method for hairy root transformation of plants described in Example 2.

The target host plant, M. truncatula was transplanted into the conventional flowerpot in which C. zizanioides was planted as in Example 2, and ½ to ¾ of the aerial parts of the C. zizanioides were subtracted with scissors to reduce the competitive growth potential of the C. zizanioides, and the co-cultivation was performed for one month. The method described in Example 3 was then used to verify that the AM fungal gene of the target host plant was over-expressing in the fungi.

The results were shown in FIG. 4 and the results was observed by the fluorescence microscope. FIG. 4a was a graph showing a signal of Green Fluorescent Protein (GFP) in the living mycorrhizal symbiotic root system. FIG. 4b was a graph showing a position of the arbuscule structure confirmed by AF488WGA fluorescent dye (NANJING WARBIO, Wheat Germ Agglutinin Alexa Fluor 488Alexa Fluor 488, Cat. No. DU-035). No green fluorescent signal existing in the symbiotic arbuscule structure of the living mycorrhizal symbiotic root system was confirmed according to the corresponding position of the arbuscule structure in FIG. 4a and FIG. 4b, that was, the green fluorescent protein was not highly expressed in the arbuscule structure.

Combining Example 3 with Comparative Example 1, the results demonstrate that host-inducing over-expressing of the fusion gene of RiEFα gene and green fluorescent protein gene in the fungi can be achieved using the device and method of the present application

### LISTING OF REFERENCE SIGNS

1. mycorrhizal chamber;
2. hyphal chamber;
101. nylon mesh;
102. support mesh;
103. hyphal holes;
201. support column.

What is claimed is:

1. A device for host-inducing over-expressing of arbuscular mycorrhizal fungal genes, comprising an upper layer and a lower layer, wherein the upper layer is a mycorrhizal chamber, the lower layer is a hyphal chamber; a bottom of the mycorrhizal chamber is defined with a plurality of hyphal holes through which hyphae reaches the hyphal chamber to absorb nutrients, a support mesh is provided on the plurality of hyphal holes, a nylon mesh is provided on the support mesh, and a pore size of the nylon mesh allows the hyphae to pass through but limits passage of a plant root system.

2. The device according to claim 1, wherein the pore size of the nylon mesh is 40 to 45 μm.

3. The device according to claim 1, wherein a pore size of the support mesh is 0.5 to 0.8 cm.

4. The device according to claim 1, wherein a pore size of each of the plurality of hyphal holes is 2 to 5 cm.

5. The device according to claim 1, wherein a support column for supporting the mycorrhizal chamber is provided in the hyphal chamber.

6. A method for accelerating nutrients exchange between arbuscular mycorrhizal fungi and a target host plant at a symbiotic interface and for achieving host-induced over-expressing of the arbuscular mycorrhizal fungal genes in the arbuscular mycorrhizal fungi based on a Host-Induced Gene Silencing (HIGS) strategy using the device according to claim 1, comprising the following steps:
S1, adding a sterilized substrate into the mycorrhizal chamber of the device, and adding a nutrient solution for hyphae growth into the hyphal chamber;
S2, planting nurse plants in the mycorrhizal chamber and inoculating an arbuscular mycorrhizal fungal inoculum, watering a nutrient solution for plant growth into the mycorrhizal chamber, and performing symbiotic co-culture to establish a stable symbiotic relationship between the arbuscular mycorrhizal fungi and the nurse plants, and thereby achieving acceleration of nutrient exchange in the stable symbiotic relationship between the arbuscular mycorrhizal fungi and the nurse plants; and
S3, using a hairy root transformed plant with over-expressing arbuscular mycorrhizal fungal genes as the target host plant, transplanting the target host plant to the mycorrhizal chamber, and subtracting ½ to ¾ of aerial parts of the nurse plants with scissors, performing symbiotic co-culture to establish a symbiotic relationship between the target host plant and the arbuscular mycorrhizal fungi, and enabling messenger ribonucleic acid (mRNA) molecules over-expressed in a root system of the target host plant to pass through an arbuscule interface and accumulate and express in the arbuscular mycorrhizal fungi in an excessive amount during a process of nutrients exchange at the arbuscule interface between cells in the root system of the target host plant and the arbuscular mycorrhizal fungi.

7. The method according to claim 6, wherein the nutrient solution for plant growth in the step S2 is a low-phosphorus nutrient solution, a phosphate concentration of the low-phosphorus nutrient solution is 80 to 120μM, and a pH value of the low-phosphorus nutrient solution is 5.5-5.7.

8. The method according to claim 6, wherein the nutrient solution for hyphae growth in the step S1 is a high-phosphorus nutrient solution, a phosphate concentration of the high-phosphorus nutrient solution is 1 to 3 mM, and a pH value of the high-phosphorus nutrient solution is 5.1-5.3.

9. The method according to claim 6, wherein in the step S2 and the step S3, conditions of the symbiotic co-culture are: culturing under a light intensity of 24000 to 26000 lux and ensuring water required for growth.

10. The method according to claim 6, wherein a liquid level of the nutrient solution for hyphae growth in the step S1 is 1 to 2 cm lower than the bottom of the mycorrhizal chamber.
